(19) United States
(12) Patent Application Publication
(10) Pub. No.: US 2016/0058060 A1
(43) Pub. Date: Mar. 3, 2016

(54) FOOD SALT PRODUCT

(71) Applicant: SMART SALT INC, Arnold, CA (US)

(72) Inventors: Leif RAMM-SCHMIDT, Espoo (FI); Helen MITCHELL, Birchington (GB); Tero HUOPANIEMI, Helsinki (GB)

(73) Assignee: SMART SALT INC, Arnold, CA (US)

(21) Appl. No.: 14/783,609

(22) PCT Filed: Apr. 10, 2014
(86) PCT No.: PCT/FI2014/050258

§ 371 Date: Oct. 9, 2015

(30) Foreign Application Priority Data
Apr. 10, 2013 (FI) 20130102

(51) Int. Cl. A23L 3/358; A23B 4/023; C01F 11/24; A61K 33/06; C01F 5/30; A23L 1/237; A23B 4/027

(52) U.S. Cl. CPC A23L 3/358; A23L 1/237; A23B 4/023; A23B 4/027; A23L 1/2375; A61K 33/06; C01F 5/30; C01F 11/24; A23V 2002/00

(57) ABSTRACT

The invention provides a multi-component homogenous co-crystallized low sodium salt product for food and pharmaceutical use. The salt product of the invention is essentially segregation-free, has low hygroscopicity and is free-flowing. It has good microbial depression properties and good taste. It supplies the functionality of salt (NaCl) in processed foods and it also maintains the microbial safety, nutritional value and taste. The salt product of the invention includes an alkaline and alkaline earth metal chloride component and an ammonium chloride component. An alkaline metal is potassium (K), and optionally also sodium (Na). An alkaline earth metal is Magnesium (Mg) or Magnesium (Mg) and Calcium (Ca) having the sum of the molar ratios 1. The invention provides also a process to produce the salt products of the invention.

### FIELD OF THE INVENTION

This invention relates to a multi-component physiological food salt product with low sodium content and a method to produce the food salt product. The salt of the invention relates to segregation problems, anti-microbial, hygroscopicity, free-flowing properties, and taste properties. It also relates to nutritional retention of phytochemicals, vitamins and minerals in cooked vegetables. The invention relates also to the use of the salt product prepared according to the method.

### BACKGROUND OF THE INVENTION

One effect of salt (NaCl) in food use is to preserve the products and slow down growth of micro-organisms. The antimicrobial activity of salt relates largely to its effect on lowering water activity (aw) but the ability of microorganisms to tolerate salt stress in otherwise optimal conditions varies widely between species. Salt (NaCl) is commonly used throughout the food industry in processed foods for its taste, technological and preservation qualities. In fact, 75% of dietary sodium intake is from processed foods. The amount of dietary salt consumed is an important determinant of blood pressure levels and hypertension risk. High blood pressure is responsible for 13% of deaths globally. This relationship is direct and progressive with no apparent threshold and salt reduction in individuals is an important intervention in reducing blood pressure and reducing the global impact of cardiovascular disease. There is a strong movement by governmental authorities to reduce the salt content in food considerably in order to reduce dietary sodium intake from salt to recommended levels. This reduction of salt (NaCl) may cause a risk of microbial contamination and spoilage of the food. As it is undesirable to solve the problem with use of general anti-microbial agents, new solutions are needed that supply the functionality of salt (NaCl) in processed foods but that also maintain the microbial safety, nutritional value and taste.

The importance of mineral balance (sodium, magnesium, calcium, potassium) in the human diet has got increasing attention over the last years. In particular magnesium is important as this mineral is not consumed in sufficient amounts. Oral intake of magnesium as food supplement (drugs) is tremendously increasing.

Magnesium is involved in about 300 biochemical reactions in the body and plays an important role in the body's metabolism, including muscle tension, the regulation of blood pressure and bone cell function. There is an increased interest in the role of magnesium in preventing and managing disorders such as hypertension, cardiovascular disease and diabetes.

Documented health effects of magnesium include: increased bone mass, improved muscle health, reduced muscle cramps, reduced hypertension, reduced migraine attacks, reduced cardiac arrhythmias, aid in absorption of potassium and calcium, important during pregnancy etc. It is furthermore known that the uptake of calcium in the body is limited unless also magnesium is present.

It has been suggested that a substantial number of adults in the United States fail to achieve the recommended daily allowances (males 400-420 mg/day and females 310-320 mg/day). According to The National Diet & Nutrition Survey (NDNS) 2003, 50% of men and 72% of women did not meet the dietary recommendations for magnesium.

In order to be available to the body a metal ion needs to be completely dissociated from its anion. The solubility of salts is linked very closely to their stability constants in water. The higher the stability constant the less ionized the salts are in solution. Magnesium chloride is totally soluble in aqueous solution with a stability constant of zero.

Not all types of magnesium deliver the same recognizable benefits. Like other minerals of nutritional value, magnesium occurs as various inorganic and organic forms in nature. Each of these forms has varying degrees of efficiency in human biochemistry. Choosing a highly soluble form of magnesium brings both high potency and superior benefits towards health.

Magnesium chloride, which contains 12% elemental magnesium, has a stability constant of zero and is completely ionized across a large pH range, from 2, found in stomach acid, to 7.4, found in extracellular tissues such as blood and lymph. Magnesium chloride has the chloride part of its compound to produce hydrochloric acid in the stomach and enhance its absorption. This is particularly suitable for anybody with low stomach acid. Compare this to magnesium sulphate which contains 10% elemental magnesium and also known as Epsom salts. Bioavailability is limited and variable with degrees of mild diarrhea depending on dosage.

This indicates that mineral balance is important not only for nutritional quality of the diet and subsequent health but also for the all-important taste experience, preservation and functionality in food products.

Salt (NaCl) for food use has been fully or partly replaced by other mineral chlorides and sulfates (e.g. CaCl₂, MgCl₂, KCl, K₂SO₄ and MgSO₄) to produce so called “physiological health salts” or “mineral salts”. Further, it has been reported that the divalent chlorides (CaCl₂ or MgCl₂) in particular perform very well as anti-microbial substances against certain bacterial species, often better than salt (NaCl). The problem with such replacements is the impact on the taste profile of food products typically leading to a bitter or metallic taste. Handling these salts is particularly difficult in a food processing environment as they are extremely hygroscopic and tend to clump together. A simple heterogeneous salt blend with these chlorides will strongly absorb moisture from the surrounding air, and cause the salt blend to humidify and eventually cake. A humidified salt is not free-flowing and causes handling problems in industrial dosage equipment. A low value of Equilibrium Relative Humidity (ERH) indicates the propensity of a product to pick-up moisture from the environment. The ERH of salt (NaCl) at room temperature is 74% whilst that of magnesium chloride is 32.8% and for calcium chloride it is still lower, but the value cannot be exactly determined.

Magnesium sulphate (“Epsom salt” MgSO₄) is less hygroscopic but has been reported to be very poor from an antimicrobial point of view. It has an extremely bitter taste and can therefore not be used in any higher degrees to replace salt (NaCl) in physiological health salts due to taste issues.

In order to reduce the hygroscopicity of magnesium chloride, it has been crystallized together with (1) ammonium chloride to form a homogenous double salt (U.S. Pat. No. 6,787,169) or (2) potassium and ammonium chloride to form a triple salt with molar ratios MgCl₂=1, KCl+NH₄Cl=1 (WO 2009/117702 A2) having Formula (i)

MgKₓ(NH₄)_{y}Cl_{g}·zH₂O (i)

wherein x+y=1, and 0≦x≦1 and 0<y≦1; g=3 and z=4-6.

In these patent publications the final salt mixture for use in food products is produced by blending the double or triple salt containing magnesium chloride with selected amounts of potassium chloride (KCl) and salt (NaCl) to form a heterogeneous blend of three salt ingredients. The double or triple salt component, if used alone, has been shown to have a somewhat bitter and metallic taste but in combination with salt (NaCl) the taste is acceptable. Thus the double or triple salt component is generally not used alone in food products and a combination with salt (NaCl) is recommended for optimal taste. The problem with this combination of salts is the risk of segregation and uneven distribution of the different ingredients/components, which is further pronounced by the fact that the salt crystals used for the blending procedure (the double or triple salt, potassium chloride and salt (NaCl)) have very different specific weights. To minimize this undesired effect the crystal size of each component should be equal. This leads to a further problem in sourcing the raw materials for the blend (potassium chloride and salt (NaCl)), as it is not always easy to find commercial salts with the correct crystal size. This easily leads to an uneven product and potential taste issues.

By measuring the equilibrium relative hygroscopicity value (ERH-%) of various homogenous salt compositions according to Formula (i) (WO 2009/117702 A2) it has also been shown that the closer the molar ratio of ammonium chloride is to 1, the lower is the humidity absorption of the magnesium ingredient with a corresponding change in ERH to a higher value. A co-crystal without ammonium chloride (pure potassium carnallite) or with very low ammonium chloride content is not practical any more for use in a salt blend based on magnesium chloride, and will show almost similar humidification and handling problems as pure magnesium chloride.

Heterogeneous salt mixtures or dry blends disclosed as salt (NaCl) replacers in WO 2009/117702 feature segregation problems which can lead to bitter taste as the different salt crystals may be unevenly distributed in the product. This may be due to insufficient blending, segregation in the packaging machine, vibration during transport, or even simply when pouring out the salt from a bag or container. In particular when the product is used without dissolving and sprinkled on snack foods (chips, French fries, peanuts, popcorn) problems of uneven distribution will develop. A heterogeneous product used in dry form does not taste as good as the taste buds of the tongue can distinguish the taste of single crystals even if the distribution of the individual crystals in the heterogeneous salt product appears to be good.

The heterogeneous salt mixtures disclosed in WO 2009/117702 also need rather high proportions of ammonium chloride in order to avoid humidification of the salt product in normal conditions. The use of ammonium chloride in higher amounts in food products is problematic because of use limit levels and declaration issues and is thus a less desirable solution.

It is also generally known, that it is not easy to crystallize together different types of alkali and/or alkaline earth metal salts. Potassium chloride or ammonium chloride can, under certain conditions, crystallize together with magnesium chloride to form uniform co-crystals called potassium carnallite and ammonium carnallite. In these double salts the molar ratio is typically 1:1. Co-crystallization with sodium chloride (NaCl) is difficult as the solubility of sodium chloride is much lower and it tends to crystallize out first and may stay in the salt slurry as more or less pure individual salt crystals. Calcium chloride is more soluble than magnesium chloride and will crystallize last.

If carbonates or sulfates are present in the solution calcium will precipitate out at an early stage as calcium carbonate or gypsum (calcium sulphate) as is found in commercial sea salt production by solar evaporation.

In order to reduce the above mentioned problem of segregation of the individual salts in a physiological salt product, different techniques to crystallize the double salts carnallite (KMgCl₃·6H₂O) and kainite (KClMgSO₄·3H₂O) together with salt (NaCl) have been presented (WO 90/00522 A1). In this publication the role of ammonium chloride in the products containing magnesium chloride has not been realized. Hence the salt products of this publication are expected to be very hygroscopic even at normal room conditions, and will not be of practical use due to humidification, low flowability and potential caking of the product. Furthermore, products having high amounts of magnesium sulphate are expected to have bitter taste, reduced microbial depression properties and being less desired from a physiological point of view. It is also known that no commercial salt products corresponding to this publication are available in the market.

The crystallizing techniques of this publication are, however, not very practical as separation of mother liquor from a crystal slurry means that the crystal mass has a different composition to the separated mother liquor and the salt product does not exactly correspond to the initial recipe. The wet salt product is finally dried in a separate dryer thus introducing additional cost of investment and production. This publication thus fails to teach crystallizing techniques by which a free-flowing salt product can be afforded in a single reactor.

This publication also includes a technique where a dry crystal cake of salt is crushed and screened to get the final salt product. This step may produce individual particles of slightly different composition as the micro crystals of salt (NaCl) attached as a layer on top of the core crystal are mechanically ripped off from the conglomerate. Also, dust problems and recirculation of off-spec products means additional production costs.

### BRIEF DESCRIPTION OF THE INVENTION

The present invention provides a salt product with low sodium content by which segregation can be greatly decreased or even fully eliminated. This can be achieved by co-crystallizing additional potassium chloride (KCl) and even a sodium chloride (NaCl) component with an alkaline earth metal and an alkaline metal component(s) and an ammonium chloride component to form multi-component salt products of the invention.

It has now also been invented that increasing the potassium chloride content in the co-crystallized salt product of the invention, including an earth metal chloride like magnesium chloride, will have a similar effect on the hygroscopic properties as by increasing the ratio of ammonium chloride. It is preferred to increase the potassium chloride content far beyond the molar ratios given in WO 2009/117702. In the salt product according to the invention a molar ratio of the potassium chloride content to magnesium chloride can be even 1.2-8× magnesium chloride.

Use of an ammonium chloride component in the co-crystallization is still beneficial for at least two identified reasons but in this way it is possible to keep the ammonium chloride content at a low level which is acceptable with respect to use limits and declaration issues. Ammonium chloride at these levels can be declared as processing aid.

It has been found out that by special crystallizing methods it is possible to create homogenous salt products where the different alkali- and alkaline earth metal salts are connected by covalent or other strong chemical bonds.

In order to distinguish between different ways of forming salt compositions it is essential to clarify some basic concepts:

A heterogeneous salt product refers to a dry salt blend of two or more crystalline salts. The individual salts are not bonded to each another in any way and can be separated by simple mechanical means (e.g. vibration, sieving etc.). Thus these salt products are subject to segregation in handling and storage.

A homogenous salt product on the other hand refers to a double, triple, quadruple or even higher salt product, wherein the salt molecules are distributed regularly in the crystal lattice (as in e.g. carnallite) and cannot be separated by simple mechanical means, i.e. product is essentially segregation free. But also double, triple, quadruple or even higher salt products, where the individual salt molecules are bonded to each another in any manner, and may be irregularly distributed in the crystal lattice or partly or fully as conglomerates of crystals attached to each another or as layers so that they cannot be separated by simple mechanical means are called homogenous salt products.

Co-crystallization is a process where the individual salt components are combined together by crystallization to form a homogenous salt product, which is essentially segregation free.

A multi-component salt refers to any salt product composed of more than one alkaline and/or alkaline earth mineral salt.

At room conditions non-hygroscopic salt product refers to a salt product, which does not absorb humidity from the surrounding when stored in a room (private home, warehouse or food production factory etc.) having a relative humidity level of about 50-65% and a temperature of 20-25° C. Very rarely indoor conditions will exceed this humidity level. The point where a salt starts to absorb humidity from the surrounding can be measured by a hygrometer using standard methods. The equilibrium value for the salt product is expressed as ERH-% (Equilibrium Relative Humidity).

Free-flowing refers to something that is able to move without anything stopping it. A free-flowing material or a substance such as a salt product has the ability to flow out in a continuous stream from a bag, dosing machine, dispenser or equal without clogging. It has good flowability. A humid or humidified salt product is not considered free-flowing.

### DETAILED DESCRIPTION OF THE INVENTION

The present invention provides a homogenous co-crystallized salt product for food use. The said salt product has good microbial depression properties and is free-flowing and segregation free. The said salt product includes an alkaline earth metal chloride component, at least one alkaline metal chloride component, an ammonium chloride component and optionally second alkaline metal chloride component and has a general Formula (I)

MgₐCa_{b}K_{c}(NH₄)_{d}NaₑCl_{f}·zH₂O (I)
wherein
a+b=1, 0<a≦1, 0≦b 21 1,
1.2≦c≦8,
0<d≦1,
0≦e≦20,
3.2≦f≦30,
and
z represents water of crystallization and is in the range of 2-6, in particular z is 4-6.

The homogenous co-crystallized 3-, 4-, or 5-salt product according to the present invention can contain sodium chloride (NaCl) and also calcium chloride (CaCl₂) in various amounts. The salt of Formula (I) of the present invention has good hygroscopic properties despite of a high proportion of magnesium chloride. The co-crystallized homogenous salt of the invention has a purer salt taste than a heterogeneous mix of the said components when tasted in dry form or used as a topping or applied by sprinkling in food applications.

It has been found out that a certain level of ammonium chloride is preferred in these salt compositions as it is very efficient in reducing the humidity absorption of magnesium and calcium chloride. The best result is achieved when the used NH₄-level is as high as possible with respect to the use limits and declaration issues.

In crystallizing tests it has now been found out that ammonium chloride also enhances the formation of homogenous triple salt products and also 4- and 5- alkali- and alkaline earth metal salt products according to the invention.

The salt product of the invention may preferably contain different components in following ratios (while a+b=1):
calcium (Ca) in molar ratio (b) 0≦b<1, preferably 0-0.5, more preferably 0-0.25;
potassium (K) in molar ratio (c) of 1.2-8, preferably 2-6, more preferably 3-4,
sodium (Na) in molar ratio (e) 0-20, preferably 5-15, more preferably 8-12; and
ammonium (NH₄) in molar ratio (d) of more than 0 to 1, preferably 0.1-0.75, more preferably 0.25-0.5.

As this co-crystallized salt product of this invention is homogenous, it solves the problem of segregation. Also, a separate blending operation is not needed (as in connection of the salts disclosed in U.S. Pat. No. 6,787,169 and WO2009/117702), hence savings in production costs are achieved.

In an embodiment of the invention the homogenous salt does not contain sodium, i.e. e is 0. Such sodium free salt product has good microbial depression properties and is free-flowing and segregation-free. It is also non-hydroscopic in at room conditions. Such sodium free salt product can be used as such or in combination with NaCl in food products.

In typical embodiment of the invention a=about 0.75; b=about 0.25; c=about 4; d=about 0.5; e=about 9; f=about 15.5; and z=about 5. In another typical embodiment of the invention a=1; b=about 0; c=about 4; d=about 0.1; e=about 0; f=about 5.1; z=about 6.

In several tests it has been proved that the salt products according to this invention are more effective in depressing microbial activity in food products than equal amount of regular salt. The higher the content of magnesium and calcium chloride is the better is the effect. This invention makes it possible to increase the usage levels more than with previous methods.

Cooking tests with vegetables have indicated that the presence of salt products according to this invention in the cooking liquor retained chlorophyll content much better than regular salt (NaCl) samples. Magnesium is situated in the center of the structure of chlorophyll and the presence of magnesium in the salts helps to prevent the loss of magnesium in the chlorophyll structure. This invention indicates the usefulness of this salt product as a means of maintaining the color of vegetables and their nutrient/mineral content.

The salt products according to this invention can, because of the segregation free properties, good taste and microbial safety, beneficially be used to partly or completely replace salt (NaCl) in particular in topical applications (pea nuts, salt sticks, French fries, popcorn etc.), but also in any food and drink applications (processed meat, vegetable, dairy, and bakery products, sports drinks and other products) as well as in pharmaceutical application products to improve the microbial properties, safety, and shelf life of said food and pharmaceutical products. It is also ideal for household use as in dispensers and for any home cooking. It can also be used to replace salt or mineral salt in spice blends and seasoning salt mixtures.

The homogenous co-crystallized salt products according to this invention achieving covalent or other strong chemical bonds between the different components are produced technically by dissolving the salts partially or completely in water, typically in a separate vessel or in the crystallizer itself, feeding the partially or completely dissolved salt fractions in right proportion and order to the crystallizer and totally removing the water phase by evaporation, typically either in atmospheric or vacuum conditions, and drying, typically in the same crystallizer, until dryness, in particular until total dryness, to afford a free-flowing salt product. The present invention may also include continuous or discontinuous feeding of a certain component to the reactor during the crystallization process in order to get a salt product with a crystal structure that is as homogenous as possible. Total removal of the water from the solution mixture means that the final salt product corresponds exactly to the initial recipe. Typical vessels for performing total drying are vacuum vessels equipped with a heat jacket and powerful, but still gentle mixing devices. All steps, i.e. dissolving, evaporation, crystallization and total drying, required to create a free-flowing salt product of the invention can be done in a single vessel, thus also saving in investment costs and processing labor.

According to the invention, using ammonium chloride (NH₄Cl) in the recipe further enhances the formation of homogenous crystals with lesser amounts of conglomerates. This has a beneficial effect on the drying and the free-flowing properties of the salt product as it facilitates the drying stage and slows down the humidification of the product when exposed to humid air.

Conventional crystallization process with centrifugation of the slurry to remove remaining mother liquor and drying of the salt product in a separate dryer is inferior because the individual components have different solubility and start to crystallize out in different order based on solubility under the current conditions. That means that the mother liquor composition differs from the solid crystal composition and it is difficult to get a salt which corresponds to the given recipe. In addition, the individual salts may partly stay in the slurry as rather pure free crystals, which after drying can be separated by simple mechanical means (sieving and vibration). Also a variation of the process conditions (temperature, pressure, pH) will generally produce salt products with different compositions because the individual salt components have a different temperature and pH dependence on solubility. See Table 1 for aqueous solubility values.

TABLE 1
Aqueous solubility of different chlorides as function of boiling temperature
(mass-% of solute). Source: CRC Handbook of Chemistry and Physics,
84th Edition, edited by David R. Lide

| | Temperature, ° C. | | | | | |
|---|---|---|---|---|---|---|
| Compound | 0 | 20 | 40 | 60 | 80 | 100 |
| CaCl₂ | 36.70 | 42.13 | 52.85 | 56.73 | 58.21 | 59.94 |
| MgCl₂ | 33.96 | 35.58 | 36.77 | 37.97 | 39.62 | 42.15 |
| NH₄Cl | 22.92 | 27.27 | 31.46 | 35.49 | 39.40 | 43.24 |
| KCl | 21.74 | 25.39 | 28.59 | 31.40 | 33.86 | 36.05 |
| NaCl | 26.28 | 26.41 | 26.67 | 27.03 | 27.50 | 28.05 |

By using a total drying process according to the invention these problems of the prior art can be overcome and it is possible to produce free-flowing homogenous salt products in a single step, where the individual salt components cannot be separated by simple mechanical means (vibration or sieving).

Following examples describe some of the embodiments of the invention.

### EXAMPLES

### Example 1

Production of a homogenous sodium free and free-flowing high potassium crystalline triple salt with low humidity absorption. 203.3 g (1 mol) MgCl₂·6H₂O, 298.2 g (4 mol) KCl, and 26.7 g (0.5 mol) NH₄Cl were dissolved totally in an open vessel in about 700 ml water by heating to boiling. The free water phase was completely removed from the solution mixture by evaporating and drying and a composition that exactly corresponds to the recipe of Formula (I) was received:

MgK₄(NH₄)_{0.5}Cl_{6.5}·6H₂O

The white, homogenous, free-flowing crystalline product of 528 g had pleasant salty taste and an ERH value of 60%. It maintained its free-flowing characteristics when exposed to ambient air at normal room conditions. The product could be used as such to replace up to 50% of salt (NaCl) in food preparations.

### Example 2

Production of a homogenous sodium free and free-flowing high potassium crystalline triple salt with low humidity absorption using batch addition of KCl. The purpose of this example is to show the effect of batch addition of part of the potassium chloride component.

203.3 g (1 mol) MgCl₂·6H₂O, 149.1 g (2 mol) KCl, and 26.7 g (0.5 mol) NH₄Cl were dissolved totally in a vessel in about 500 ml water by heating to boiling. An additional 149.1 g (2 mol) of KCl was dissolved in 240 ml water in a separate vessel and added as a single batch to the boiling crystal slurry at a point, when about 200 ml of the water had boiled off. The free water phase was completely removed from the solution mixture by evaporating and drying and a composition that exactly corresponds to the recipe of Formula (I) was received:

MgK₄(NH₄)_{0.5}Cl_{6.5}·6H₂O

The white, homogenous, free-flowing crystalline product of 528 g had pleasant salty taste and an ERH value of 62%. It maintained its free-flowing characteristics when exposed to ambient air at normal room conditions. The product could be used as such to replace up to 50% of salt (NaCl) in food preparations.

### Example 3

Production of a homogenous sodium free and free-flowing high potassium crystalline triple salt with low humidity absorption using continuous addition of KCl. The purpose of this example is to show the effect of continuous addition of part of the potassium chloride component.

203.3 g (1 mol) MgCl₂·6H₂O, 149.1 g (2 mol) KCl, and 26.7 g (0.5 mol) NH₄Cl were dissolved totally in a vessel in about 500 ml water by heating to boiling. An additional 149.1 g (2 mol) of KCl was dissolved in 240 ml water in a separate vessel and added continuously to the boiling crystal slurry, starting at a point, when about 100 ml of the water had boiled off. The free water phase was completely removed from the solution mixture by evaporating and drying and a composition that exactly corresponds to the recipe of Formula (I) was received:

MgK₄(NH₄)_{0.5}Cl_{6.5}·6H₂O

The white, homogenous, free-flowing crystalline product of 528 g had pleasant salty taste and an ERH value of 62%. It maintained its free-flowing characteristics when exposed to ambient air at normal room conditions. The product could be used as such to replace up to 50% of salt (NaCl) in food preparations.

### Example 4

Production of a homogenous sodium free crystalline triple salt with low ammonium chloride content and moderate humidity absorption. The purpose of this example is to show the effect of reduced ammonium chloride content.

203.3 g (1 mol) MgCl₂·6H₂O, 149.1 g (2 mol) KCl, and 5.3 g (0.1 mol) NH₄Cl were dissolved totally in a vessel in about 650 ml water by heating to boiling. An additional 149.1 g (2 mol) of KCl was dissolved in 240 ml water in a separate vessel and added as a single batch to the boiling crystal slurry at a point, when about 200 ml of the water had boiled off. The free water phase was completely removed from the solution mixture by evaporating and drying and a composition that exactly corresponds to the recipe of Formula (I) was received:

MgK₄(NH₄)_{0.1}Cl_{6.1}·6H₂O

The white, homogenous crystalline product of 507 g had pleasant salty taste and an ERH value of 55%. It maintained it's free-flowing characteristics rather well when exposed to ambient air at normal room conditions, but not as well as Example 1. The product could be used as such to replace up to 50% of salt (NaCl) in food preparations.

### Example 5

Production of a homogenous sodium free crystalline triple salt with moderate potassium chloride and low ammonium chloride content and moderate humidity absorption. The purpose of this example is to show the effect of reduced potassium chloride content in comparison to Example 2.

203.3 g (1 mol) MgCl₂·6H₂O, 149.1 g (2 mol) KCl, 5.3 g (0.1 mol) NH₄Cl were dissolved totally in a vessel in about 650 ml water by heating to boiling. The free water phase was completely removed from the solution mixture by evaporating and drying and a composition that exactly corresponds to the recipe of Formula (I) was received:

MgK₂(NH₄)_{0.1}Cl_{4.1}·6H₂O

The white, homogenous crystalline product of 358 g had pleasant salty taste and an ERH value of 53%. Due to the very low ammonium chloride content in combination with rather low potassium chloride content, it did not maintain its free-flowing characteristics as well as e.g. the product of Example 1 and 2 when exposed to ambient air at normal room conditions and gradually lost its free-flowing characteristics. The product could be used as such to replace up to 50% of salt (NaCl) in food preparations.

### Example 6

Production of magnesium potassium carnallite with high humidity absorption. The purpose of this example is to show the effect of omitting the ammonium chloride content totally and low content of potassium chloride. 146.4 kg MgCl₂·6H₂O and 53.6 kg KCl (molar ratio 1:1) were totally dissolved in 150 l water and crystallized in a vacuum reactor. The free water phase was completely removed from the solution mixture by evaporating and drying and a composition that exactly corresponds to the recipe of carnallite was received:

MgKCl₃·6H₂O

The white, homogenous crystalline product of 200 kg had a slightly bitter salty taste and an initial ERH value of 37% increasing gradually to 47% where it stabilized. When exposed to ambient air at normal room conditions, the product soon lost its free-flowing characteristics, and later caked.

### Example 7

Production of a homogenous 51% sodium reduced free-flowing crystalline 4-salt salt with low humidity absorption. 203.3 g (1 mol) MgCl₂·6H₂O, 298 g (4 mol) KCl, 40.1 g (0.75 mol) NH₄Cl and 526 g (9 mol) NaCl were dissolved totally in a vessel in about 1800 ml water by heating to boiling. The free water phase was completely removed from the solution mixture by evaporating and drying and a composition that exactly corresponds to the recipe of Formula (I) was received:

MgK₄(NH₄)_{0.75}Na₉Cl_{15.75}·6H₂O

The white, homogenous free-flowing crystalline product of 1068 g had pleasant salty taste and an ERH value of 61%. It maintained its free-flowing characteristics when exposed to ambient air at normal room conditions. The product could be used as such to replace up to 100% of salt (NaCl) in food preparations.

### Example 8

Production of a homogenous 50% sodium reduced, free-flowing crystalline 4-salt salt with and free-flowing low humidity absorption.

29.1 kg MgCl₂·6H₂O, 40.2 kg KCl, 5.7 kg NH₄Cl (molar ratio 1:4:0.75) were dissolved totally in about 120 l water by heating to boiling and crystallized in a vacuum reactor. 75 kg NaCl (molar ratio 9) was dissolved in 205 l water in a separate vessel, and fed to the reactor continuously at a rate of 1 l/min starting at a point when 50 l water had boiled off. The free water phase was completely removed from the solution mixture by evaporating and drying and a composition that exactly corresponds to the recipe of Formula (I) was received:

MgK₄(NH₄)_{0.75}Na₉Cl_{15.75}·6H₂O

The white, homogenous, free-flowing crystalline product of 150 kg had pleasant salty taste and an ERH value of 61%. It maintained it's free-flowing characteristics when exposed to ambient air at normal room conditions. The product could be used as such to replace up to 100% of salt (NaCl) in food preparations.

### Example 9

Production of a homogenous 50% sodium reduced free-flowing crystalline 5-salt salt with moderate calcium chloride content and with low humidity absorption.

152.5 g (0.75 mol) MgCl₂·6H₂O, 36.8 g (0.25 mol) CaCl₂*2H₂O, 298 g (4 mol) KCl, 40.1 g (0.75 mol) NH₄Cl and 526 g (9 mol) NaCl were dissolved totally in an open vessel in about 1800 ml water by heating to boiling. The free water phase was completely removed and a composition that exactly corresponds to the recipe of Formula (I) was received:

Mg_{0.75}Ca_{0.25}K₄(NH₄)_{0.75}Na₉Cl_{15.75}·5H₂O

The white, homogenous free-flowing crystalline product of 1054 g had a slightly bitter, but still acceptable salty taste and an ERH value of 57%. It maintained its free-flowing characteristics when exposed to ambient air at normal room conditions.

### Example 10

A salt sample prepared according to Example 7 of the invention was used to test the microbial growth/survival of L. monocytogenes in frankfurter samples in comparison to table salt (NaCl) at equal dosage levels and at a storage temperature of 5° C. No nitrites were added to the samples. The results indicated that although both salt types were able to support the growth of L. monocytogenes, the sample according to Example 7 was able to delay the growth of the organism over storage time. The table salt sample showed an increase of L. monocytogenes to a count of 4 Log after 23 days of storage, while the same increase for the Example 7 salt did not occur until 28 days storage. The frankfurters were subject to sensory testing. The expert taste panel was not able to distinguish any difference in flavour between the two samples. As a further benefit, the test indicated that using the salt product according to the invention, the addition of nitrites can be reduced or even omitted.

### Example 11

A salt sample according to Example 7 of the invention was used to test the microbial growth in bread. Individual dough pieces, prepared using a salt sample according to Example 7 of the invention and table salt at a salt level of 1.2% w/w in the final loaf, were inoculated with spore suspensions of a cocktail of B. cereus and B. subtilis at 107-108 spores/g of final product and baked using standard domestic bread makers. Inoculated and uninoculated loaves (controls) were microbiologically analyzed over 6 days storage at 21° C. and 25° C. Analysis was carried out on day 0 (post baking and after cooling), day 1, day 2 and day 6. The results highlighted two main differences between the two salt types. Immediately post baking (and after cooling) the bread loafs containing salt according to Example 7 of the invention showed significant log drop of up to 4.7-Logs in Bacillus spp. counts compared with a much smaller drop of up to 3.4-Log in the table salt containing breads. Although in breads containing both salt types, Bacillus spp. counts later picked up to very high levels during storage, this initial difference in lethality indicates that salt according to Example 7 of the invention in combination with the heat applied during baking contributes to a significantly increased process lethality compared to table salt.

Additionally, the results from the control, un-inoculated bread samples indicated that even though there was no difference in the yeast and mould counts over time, total aerobic viable counts in the table salt-containing bread samples significantly increased to c.10⁴-10⁵ cfu/g at the end of the storage period (Day 6) under both storage temperatures. Counts below the limit of detection were obtained throughout storage of the Example 7 salt containing control bread samples at 21° C., while small recovery (c. 10-10² cfu/g) was observed in the samples stored at 25° C. These results indicate that it is possible to extend the shelf life of bread using Example 7 salt.

### Example 12

Broccoli florets were cooked using the three different cooking methods using no salt, 1.0 g NaCl or 1.0 g of salt of Example 7. Samples were then analyzed for their antioxidant capacity using the FRAP assay and their chlorophyll content was assessed using a spectrophotometric procedure. Broccoli which had been boiled, steamed or microwaved with salt of Example 7 was found to have a carotene and chlorophyll content significantly higher than broccoli cooked using NaCl.

It will be obvious to a person skilled in the art that, as the technology advances, the inventive concept can be implemented in various ways. The invention and its embodiments are not limited to the examples described above but may vary within the scope of the claims.

1. A homogenous co-crystallized salt product for food use said salt product having good microbial depression properties and being segregation-free, said salt product including an alkaline earth metal chloride component, at least one alkaline metal chloride component, an ammonium chloride component and optionally a second alkaline metal chloride component and having general Formula (I)
MgₐCa_{b}K_{c}(NH₄)_{d}NaₑCl_{f}·zH₂O (I)
wherein
a+b=1, 0<a≦1, 0≦b<1,
1.2≦c≦8,
0<d≦1,
0≦e≦20,
3.2≦f≦30,
and
z represents water of crystallization and is in the range of 2 to 6.

2. The homogenous salt product according to claim 1, wherein e is 0.

3. The homogenous salt product according to claim 1, wherein z is 4-6.

4. The homogenous salt product according to claim 1, wherein
0≦b≦0.5;
2≦c≦6;
0.1≦d≦0.75; and
5≦e≦15.

5. The homogenous salt product according to wherein
a=about 0.75
b=about 0.25
c=about 4
d=about 0.5
e=about 9
f=about 15.5
z=about 5.

6. The homogenous salt product according to claim 1 wherein
a=1
b=about 0
c=about 4
d=about 0.1
e=about 0
f=about 5.1
z=about 6.

7. A method for preparing a homogenous salt product according to claim 1 for use in food products, comprising
bringing together a desired amount of a magnesium chloride (MgCl₂) component, optionally a calcium chloride (CaCl₂) component, a potassium chloride (KCl) component, an ammonium chloride (NH₄Cl) component, and optionally a sodium chloride (NaCl) component in a solution,
mixing and heating the solution to boiling,
optionally feeding part of the components to the solution continuously or discontinuously during the boiling process, and
completely removing the water phase of the mixture by evaporation or drying wherein the partly or fully dissolved components of the solution mixture are co-crystallized to afford the salt product of Formula (I).

8. A method for preparing a homogenous salt product according to claim 2 for use in food products, comprising
bringing together a desired amount of a magnesium chloride (MgCl₂) component, optionally a calcium chloride (CaCl₂) component, an ammonium chloride (NH₄Cl) component and a first part of a potassium chloride component in a first solution,
mixing and heating the first solution to boiling,
feeding rest of the desired part of the potassium chloride component in a second solution continuously or discontinuously during the boiling process to the first solution to form a liquid solution mixture or a slurry of the components,
completely removing the water phase of the mixture by evaporation or drying wherein the partly or fully dissolved components of the solution mixture are co-crystallized to afford the salt product of Formula (I), wherein e is 0.

9. A method for preparing a homogenous salt product according to claim 1 for use in food products, comprising
bringing together a desired amount of a magnesium chloride (MgCl₂) component, optionally a calcium chloride (CaCl₂) component, a potassium chloride component, an ammonium chloride component and a first part of a desired amount of a sodium chloride component in a first solution,
mixing and heating the first solution to boiling,
feeding rest of the desired part of the sodium chloride component in a second solution continuously or discontinuously during the boiling process to the first solution to form a liquid solution mixture or a slurry of components
completely removing the water phase of the mixture by evaporation or drying whereby the partly or fully dissolved components of the solution mixture are co-crystallized to afford the salt product of general Formula (I).

10. (canceled)

11. (canceled)

12. (canceled)

13. (canceled)

14. (canceled)

15. (canceled)

16. A food, beverage or pharmaceutical comprising the salt product of claim 1.

17. A food according to claim 16 wherein the salt product is sprinkled thereon.

18. A method of enhancing the shelf life of a food selected from bread, processed meats, fish, or dairy product, comprising providing said food containing the salt product of claim 1.

19. A method of enhancing the nutritional retention of phytochemicals, vitamins and minerals and color in said food comprising incorporating the salt product of claim 1 into said food.

20. The food, beverage or pharmaceutical according to claim 16 which additionally comprises NaCl as table salt.

21. The homogenous salt product according to claim 1, wherein
0≦b≦0.25;
3≦c≦4;
0.25≦d≦0.5; and
8≦e≦12.

22. The food according to claim 17 which is selected from peanuts, salt sticks, French fries and popcorn.
